(19) United States
(12) Patent Application Publication
(10) Pub. No.: US 2001/0001658 A1
(43) Pub. Date: May 24, 2001

(54) Granule modulating hydrogel system

(76) Inventors: Chih-Ming Chen, Davie, FL (US); Steve Jan, Coral Springs, FL (US); Jianbo Xie, Ft Lauderdale, FL (US)

Correspondence Address: James V Costigan Esq, Hedman Gibson & Costigan PC, 1185 Avenue of the Americas, New York, NY, 10036-2646, US

(21) Appl. No.: 09/490,013

(22) Filed: Jan. 21, 2000

Related U.S. Application Data
(63) Continuation of application No. 09/122,139, filed on Jul. 24, 1998.

(51) Int. Cl. A61K 9/22

(52) U.S. Cl. 424/49; 424/468

(57) ABSTRACT

A naproxen once-a-day matrix tablet is described which is based on a compressed mixture of a controlled release component of said tablet that contains:

(i) substantially homogeneous granules of naproxen or a pharmaceutically acceptable salt of naproxen and an organic acid which are prepared by a granulation process which uses a pharmaceutically acceptable film forming material as the binding material;

(ii) a pharmaceutically acceptable hydrogel forming polymer; and

(B) a coating on said compressed mixture which consists essentially of naproxen or a pharmaceutically acceptable salt thereof and a pharmaceutically acceptable film forming composition which allows for immediate release of naproxen.

### BACKGROUND OF THE INVENTION

The present invention relates to controlled release unit dose formulations of naproxen. Naproxen is sold commercially in an extended release pharmaceutical dosage form in order to maintain a therapeutic serum level of naproxen and to minimize the effects of missed doses of drugs caused by a lack of patient compliance. The minimum therapeutic plasma naproxen concentrations are in the range of 30 to 60 mcg/ml.

In the prior art, extended release formulations of naproxen tablets have been marketed which provide 24 hour therapeutic blood levels of naproxen with once a day administration of a single dosage unit. Naprelan is described as a once-a-day extended release tablet containing naproxen and fumaric acid in a system that is described as an Intestinal Protective Drug Absorption System. This system combines a rapidly disintegrating tablet system which includes an immediate release component and a pelletized sustained release component which has a membrane coating around the pellets. Example 8 of U.S. Pat. No. 5,637,320 describes a naproxen formulation which is based on the use of naproxen pellets in combination with a wet granulate of naproxen with a polymeric binder prior to compressing the mixture into a once-a-day tablet. The pellets are known in the art as reservoir devices in which a core of drug is surrounded by a polymeric membrane through which the drug is released by diffusion.

The applicants have discovered that a naproxen once-a-day formulation may be prepared without forming pellets but by preparing a wet granulate of naproxen with a polymeric binder.

The once-a-day naproxen controlled release formulation of the invention provides an alternative to the prior art formulation which requires the presence of pellets having a multilayer membrane to impart controlled release properties to a naproxen once-a-day formulation.

### SUMMARY OF THE INVENTION

The present invention provides a naproxen once-a-day matrix tablet which comprises:

(A) a compressed mixture of a controlled release component of said tablet which comprises:

(i) substantially homogeneous granules of naproxen and an organic acid which are prepared by a granulation process which uses a pharmaceutically acceptable film forming material as the binding material;

(ii) a pharmaceutically acceptable hydrogel forming polymer;

(B) a coating on said compressed mixture which consists essentially of naproxen and a pharmaceutically acceptable film forming composition which allows for immediate release of naproxen.

It is an object of the present invention to provide a once-a-day naproxen formulation.

It is also an object of the invention to provide a once-a-day tablet formulation of naproxen which can be made in a standard tabletting facility without the need to apply a multilayer membrane to the dosage formulation.

It is also an object of the invention to provide a once-a-day formulation of naproxen which contains a cellulose ether hydrogel in combination with substantially homogeneous granules of naproxen.

These and other objects of the invention will become apparent from a review of the appended specification.

### BRIEF DESCRIPTION OF THE DRAWINGS

FIG. 1 is a graph which shows the in vitro dissolution rate of naproxen sodium from the tablet of the invention. in potassium phosphate buffer, pH 7.5, using a USP Type 2 apparatus at 37° and 50 rpm.

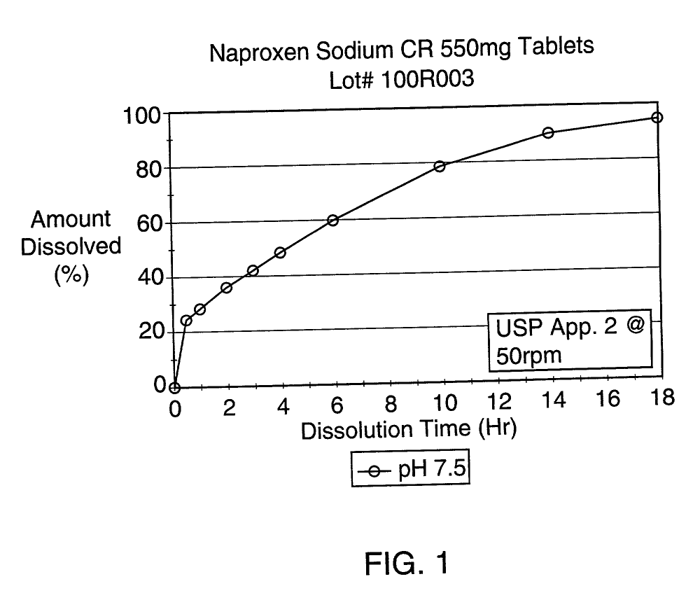

FIG. 2 is a graph which shows the in vitro dissolution rate of naproxen sodium from the extended release core tablet of the invention. in potassium phosphate buffer, pH 7.5, using a USP Type 2 apparatus at 37° and 50 rpm.

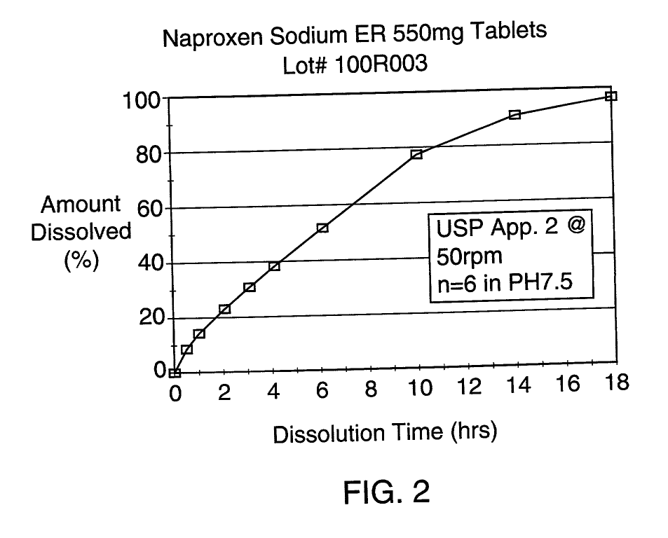

### DETAILED DESCRIPTION OF THE INVENTION

The naproxen formulation of the invention is prepared by mixing naproxen with a pharmaceutically acceptable organic acid such as fumaric, tartaric, maleic itaconic, citric, ascorbic, succinic, malic acid or mixtures thereof. The ratio of naproxen to organic acid may be from 18:1 to 1:2 and preferably about 12:1 on a weight basis. Thereafter, a compressible granulation is prepared, preferably by using a wet granulation technique. When the term naproxen is used herein it is also intended to cover pharmaceutically acceptable salts of naproxen such as the sodium salt of naproxen.

The polymer binder for the granulation may be any water insoluble pharmaceutically acceptable film forming material such as ethylcellulose, cellulose acetate phthalate, mono-glycerides such as glyceryl mono-oleate, waxes such as those disclosed in Remington's Pharmaceutical Sciences, 17th Ed., p. 405, acrylic polymers such as poly (ethylacrylate-methylmethacrylate) trimethylammonioethylmethacrylate chloride, poly(ethylacrylate-methylmethacrylate) which is commercially available as Eudragit NE30D, which is a 30 wt % aqueous dispersion of a 2:1 copolymer of ethylacrylate and methylmethacrylate having a weight average molecular weight of about 800,000, polyvinyl chloride resins, polyvinyl acetate phthalate and the like.

The granules may be made by contacting the blend of naproxen and the acid component with a water insoluble pharmaceutically acceptable film forming material. If a wax or mono-glyceride is employed, the naproxen-acid mixture may be dispersed in the molten wax or in a conventional spray congealing apparatus. It is preferred to use a wet granulation process and an acrylic copolymer to make the granules. In such a process, an aqueous dispersion of a water insoluble polymer may be added to the powders in a blender with milling or agitation depending on the particular apparatus. The powder mass is wetted to the consistency of damp snow which may then be screened through a first screen (4 to 8 mesh—US Standard) or passed through a comminuting mill to form granules which are dried on trays or in a fluid bed dryer. When the granules are dry, they are milled and passed through a 12 to 20 mesh screen (US Standard) in a suitable apparatus such as a Fitzpatrick mill to form the compressible granules that are used to make the controlled release component of the dosage form of the invention.

After the granulation is prepared, the granules are mixed with a hydrogel forming polymer which is preferably hydroxypropyl methylcellulose. Other pharmaceutically acceptable hydrogel forming polymers include carboxymethylcellulose calcium, carboxymethylcellulose sodium, guar gum, hydroxyethyl cellulose, hydroxypropyl cellulose, methylcellulose, acrylic acid crosslinked with polyalkenyl ethers or divinyl glycol (Carbomer), sodium alginate and poly(ethylene oxide) (Polyox). In addition a tablet lubricant and/or a tablet disintegrant and or glidant may be added to the mixture. Examples of suitable tablet lubricants include 0.5-5 wt % based on the weight of the compressed control release component of the tablet, of magnesium stearate or glyceryl mono-stearate. A minor amount of a pharmaceutically acceptable diluent may also be added prior to tabletting. Materials such as lactose, starch, dextrose, sucrose, hydroxypropyl cellulose, microcrystalline cellulose and the like may be added at a level of 0 to 20 wt %, preferably 5 to 15 wt %, based on the total weight of the compressed control release component. Fumed colloidal silicon dioxide may be used as the glidant at level of 0-1 wt % based on the total weight of the compressed control release component.

An immediate release coating which contains naproxen may be optionally coated directly onto the tablet core or over a sealed tablet core. A seal coat may be applied by applying a thin coating of shellac, zein, polyvinylpyrrolidone or the like with an appropriate anti-tack agent such as talc.

The immediate release coating will be applied to the compressed control release component in order to provide a finished dosage form which will have a ratio of immediate release naproxen to controlled release naproxen, based only on the total weight of naproxen, of from 1:10 to 1:1 and preferably from 1:7 to 1:2.

A coating formulation for applying an immediate release layer of naproxen to the compressed control release component may comprise a mixture of naproxen, purified water and a binder material. Generally the coating solution will comprise from 5 to 25 wt % of naproxen; from 2 to 5 wt % binder material and the balance purified water. The binder material may comprise Opadry Clear, YS-1-7006 which contains 91 wt % hydroxypropyl methylcellulose (E-6), 9 wt % polyethylene glycol which can be used as a 8-15 % w/w solution in purified water. The naproxen once-a-day matrix tablet will comprise:

(A) from 70 to 90 wt % of compressed mixture of a controlled release component of said tablet based on the total weight of the matrix tablet which comprises:

(i) 60 to 80 wt % of substantially homogeneous granules of naproxen or a pharmaceutically acceptable salt of naproxen and an organic acid, based on the total weight of the compressed mixture, which are prepared by a granulation process which uses a pharmaceutically acceptable film forming material as the binding material;

(ii) 10 to 30 wt % of a pharmaceutically acceptable hydrogel forming polymer, based on the weight of the compressed mixture;

(B) from 10 to 25 wt % of a coating on said compressed mixture, based on the total weight of the matrix tablet, which consists essentially of naproxen or a pharmaceutically acceptable salt thereof and a pharmaceutically acceptable film forming composition which allows for immediate release of naproxen.

The controlled release naproxen formulation of the invention will preferably have a dissolution release rate in a potassium phosphate buffer at pH 7.5, in a USP XXII Type II apparatus at 37° C. and 50 rpm which substantially corresponds to the following:

a) from 15 to 45 wt % and preferably from 20 to 45 wt % of naproxen is released after 2 hours;

b) from 20 to 60 wt % and preferably from 30 to 50 wt % of naproxen is released after 4 hours;

c) from 50 to 90 wt % and preferably from 60 to 80 wt % of naproxen is released after 6 hours;

d) not less than 60 wt % and preferably not less than 70 wt % of naproxen is released after 12 hours.

The invention also includes the process for preparing a once-a-day naproxen matrix tablet; said process comprising:

(a) forming a granulation of naproxen or a pharmaceutically acceptable salt thereof and a water insoluble pharmaceutically acceptable film forming polymer;

(b) milling the granulation formed in step (a);

(c) compressing the granules formed in step (b) into a tablet; and

(d) coating the compressed tablet formed in step (c) with a polymeric film forming material and naproxen or a pharmaceutically acceptable salt thereof.

### DESCRIPTION OF THE PREFERRED EMBODIMENTS

### EXAMPLE 1

A naproxen sodium compressed control release component was prepared according to the following procedure:

Stage I

Naproxen sodium (26.44 kg.) and fumaric acid (2.189 kg) were combined in a vertical granulator (FM-VG-100) with the blade speed set at 100 rpm and the cross screw set at low speed with five minutes of mixing prior to adding 11.237 g of Eudragit NE 30D and 2.9 kg of purified water over a period of 12 minutes with continued mixing. The wet granulate is discharged and oven dried at _° C. The dried granulated is then pased to a Fitzmill, operated at medium speed (1665 rpm), which has a stainless steel screen.

Stage II

127.698 kg of naproxen sodium granules prepared according to the procedure of Stage I are combined with 15.72 kg of hydroxypropyl methylcellulose USP (Methocel K4M, Premium); 10.480 kg of hydroxypropyl cellulose, NF (Klucel HXF); 18.167 kg of microcrystalline cellulose, NF (Avicel PH101). The components are blended in a slant cone blender at 17 rpm.

Stage III

172.5 kg of the blend of Stage II is combined with 0.87 kg of colloidal silicon dioxide, NF and 0.87 kg of magnesium stearate, NF to form a tabletting blend which is compressed into 0.34×0.656 capsule shaped compressed release component tablets each of which has a target weight of 728.5 mg and contains 440 mg of naproxen sodium.

Stage IV

The compressed control release component tablets are coated with an immediate release dose of naproxen sodium by coating the compressed release component tablet with a coating composition as follows:

naproxen sodium, 440 mg extended release tablets (Stage III) 82.0 kg

naproxen sodium, USP 12.259 kg

Opadry clear, YS-1-7006 1.679 kg

Purified water, USP 61.916 kg

The extended release tablets were placed in an Accela Cota and the Opadry clear was combined with the purified water prior to adding the naproxen sodium. Stirring is continued until a clear solution is obtained. The solution was coated onto the extended release tablets to form an external immediate release layer of naproxen.

While certain preferred and alternative embodiments of the invention have been set forth for purposes of disclosing the invention, modifications to the disclosed embodiments may occur to those who are skilled in the art. Accordingly, the appended claims are intended to cover all embodiments of the invention and modifications thereof which do not depart from the spirit and scope of the invention.

I claim:

1. A naproxen once-a-day matrix tablet which comprises:
(A) a compressed mixture of a controlled release component of said tablet which comprises:
(i) substantially homogeneous granules of naproxen or a pharmaceutically acceptable salt of naproxen and an organic acid which are prepared by a granulation process which uses a pharmaceutically acceptable film forming material as the binding material;
(ii) a pharmaceutically acceptable hydrogel forming polymer;
(B) a coating on said compressed mixture which consists essentially of naproxen or a pharmaceutically acceptable salt thereof and a pharmaceutically acceptable film forming composition which allows for immediate release of naproxen.

2. A naproxen once-a-day matrix tablet as defined in claim 1 wherein the organic acid is selected from the group consisting of fumaric, tartaric, maleic, itaconic, citric, ascorbic, succinic, malic acid or mixtures thereof.

3. A naproxen once-a-day matrix tablet as defined in claim 1 wherein the granules are miled prior to being compressed into tablets.

4. A naproxen once-a-day matrix tablet as defined in claim 1 wherein the pharmaceutically acceptable film forming material is selected from the group consisting of ethylcellulose, cellulose acetate phthalate, monoglycerides, waxes, acrylic polymers, acrylic acid polymers, polyvinyl chloride resins and polyvinyl acetate phthalate.

5. A naproxen once-a-day matrix tablet as defined in claim 4 wherein the pharmaceutically acceptable film forming material is a 2:1 copolymer of ethylacrylate and methylmethacrylate.

6. A naproxen once-a-day controlled release formulation as defined in claim 1 wherein the pharmaceutically acceptable hydrogel forming polymer is hydroxypropyl methylcellulose or hydroxypropyl cellulose.

7. A naproxen once-a-day controlled release formulation as defined in claim 1 which has a dissolution release rate in a potassium phosphate buffer at pH 7.5, in a USP XXII Type II apparatus at 37° C. and 50 rpm which substantially corresponds to the following:
a) from 15 to 45 wt % and preferably from 20 to 45 wt % of naproxen is released after 2 hours;
b) from 20 to 60 wt % and preferably from 30 to 50 wt % of naproxen is released after 4 hours;
c) from 50 to 90 wt % and preferably from 60 to 80 wt % of naproxen is released after 6 hours;
d) not less than 60 wt % and preferably not less than 70 wt % of naproxen is released after 12 hours.

8. A naproxen once-a-day matrix tablet which comprises:
(A) a compressed mixture of a controlled release component of said tablet which comprises:
(i) substantially homogeneous granules of naproxen sodium which are prepared by a granulation process which uses a 2:1 copolymer of ethylacrylate and methylmethacrylate as the binding material;
(ii) hydroxypropyl methylcellulose;
(B) a coating on said compressed mixture which consists essentially of naproxen sodium and a pharmaceutically acceptable film forming composition which allows for immediate release of naproxen.

9. A process for preparing a once-a-day naproxen matrix tablet; said process comprising:
(a) forming a granulation of naproxen or a pharmaceutically acceptable salt thereof and a water insoluble pharmaceutically acceptable film forming polymer;
(b) milllin the granulation formed in step (a);
(c) compressing the granules formed in step (b) into a tablet; and
(d) coating the compressed tablet formed in step (c) with a polymeric film forming material and naproxen or a pharmaceutically acceptable salt thereof.

10. A process as defined in claim 9 wherein the water insoluble pharmaceutically acceptable polymer is a 2:1 copolymer of ethylacrylate and methylmethacrylate.

11. A process as defined in claim 9 wherein the polymeric film forming material permits the naproxen or a pharmaceutically acceptable salt thereof to be released immediately after ingestion of the matrix tablet.
